(19) United States
(12) Patent Application Publication
(10) Pub. No.: US 2004/0014764 A1
(43) Pub. Date: Jan. 22, 2004

(54) N-myristoyltransferase inhibitor compositions and methods of use

(76) Inventors: Charles D. Smith, Hershey, PA (US); Kevin J. French, Harrisburg, PA (US)

Correspondence Address: Charles D. Smith, 245 Candlewyck Lane, Hershey, PA, 17033, US

(21) Appl. No.: 10/397,695

(22) Filed: Mar. 26, 2003

Related U.S. Application Data
(60) Provisional application No. 60/369,095, filed on Mar. 29, 2002.

(51) Int. Cl. A61K 31/498

(52) U.S. Cl. 514/249

(57) ABSTRACT

The invention is a pharmaceutical composition mainly for the treatment of a hyperproliferative disease, such as cancer, atherosclerosis, restenosis or psoriasis, or viral infection. The active ingredient of the composition is a compound that inhibits the activity of human N-myristoyltransferase (NMT). The NMT inhibitors are small molecule non-lipid compounds containing a cyclohexyl-octahydro-pyrrolo[1,2-a]pyrazine (COPP) or methyl-octahydro-pyrrolo[1,2-a]pyrazine (MOPP) group.

### REFERENCE TO RELATED APPLICATIONS

This application is a non-provisional application claiming priority under 35 U.S.C. section 119(e) to provisional application No. 60/369,095 filed Mar. 29, 2002, the contents of which are incorporated herein by reference.

### GOVERNMENT SPONSORSHIP

This invention was made with government support Grant CA88243 awarded by the United States Public Health Service. Accordingly, the US government may have certain rights in this invention.

### FIELD OF THE INVENTION

The present invention relates to the field of human N-myristoyltransferase inhibitors and therapeutic compositions containing the same. More specifically, the invention relates to compositions of non-lipid compounds that inhibit the enzymatic activity of N-myristoyltransferase and the use of such compositions for the treatment and/or prevention of hyperproliferative diseases, such as cancer, atherosclerosis and psoriasis, or the treatment of osteoporosis or infection by HIV or other viruses.

### BACKGROUND OF THE INVENTION

The term protein myristoylation refers to the covalent attachment of myristic acid to specific proteins. This process has been shown to target proteins to specific subcellular membranes, stabilize protein conformation, and facilitate additional lipidations (Resh, Biochim. Biophys. Acta 1451: 1 (1999)). N-Myristoyltransferase (NMT; E.C. 2.3.1.97) catalyzes this addition onto the N-terminal glycine residue of specific proteins. The reaction requires only myristoyl-CoA and a protein containing a suitable peptide sequence, and occurs through an ordered Bi Bi mechanism. The list of myristoylated proteins is diverse, including certain viral proteins, Gα proteins, tyrosine kinases of the Src family, and nitric oxide synthase. Mutation or deletion of NMT results in recessive lethality in Saccharomyces cerevisiae (Duronio et al., J Cell Biol. 113: 1313 (1991)) and Candida albicans (Weinberg et al, Mol. Microbiol. 16: 241 (1995)). Increases in NMT activity and expression have been observed in colorectal adenocarcinomas (Raju et al., Exp. Cell Res. 235: 145 (1997)), gallbladder carcinomas (Rajala et al., Cancer 88: 1992 (2000)), and the murine leukemia cell line L1210 (Boutin et al., Eur. J. Biochem. 214: 853 (1993)). Thus, NMT appears to be an important enzyme in both prokaryotes and higher organisms.

Several pathogenic states are linked to undesired NMT activity. The Rous Sarcoma Virus transforms cells through expression of the tyrosine kinase Src. Kamps et al. demonstrated that mutation of the N-terminal glycine of Src to prevent its myristoylation conserves kinase activity, but blocks its ability to associate with membranes and to transform cells (Kamps et al., Cell 45: 105 (1986)). Several additional protein tyrosine kinases of the Src family are well-established oncogenes that require myristoylation for activity (Resh. Cell 76: 411 (1994)). Importantly, a number of studies have shown that c-Src is a critical regulator of human cancers, including breast carcinomas (Garcia et al., Oncogene 20: 2499 (2001)). Src activity is increased in numerous unrelated tumors, both in elevated protein levels and increase in enzyme activity (Rosen et al., J. Biol. Chem. 261: 13754 (1986)). Correlations between Src expression and progression have been observed in colonic polyps, with the highest Src expression being concurrent with malignancy. Additionally, increases in Src activity are seen in colorectal metastases to extrahepatic sites (Termuhlen et al., J. Surg. Res. 54: 293 (1993)). Thus, the link between NMT and Src strongly implicates their association in cancer pathogenesis. Additional studies have shown that Src may be a therapeutic target for the treatment of osteoporosis (Sawyer et al., Expert Opin. Investig. Drugs 10: 1327 (2001)).

The gag gene of HIV encodes viral structural proteins produced by the processing of Pr55^{gag} (Gag). Assembly of these proteins into an active virus particle is absolutely dependent on the myristoylation of Gag. For example, mutation of the target glycine residue to an alanine residue results in accumulation of unprocessed Gag and a total blockage of virus release from cells (Bryant and Ratner, Proc. Natl. Acad. Sci. USA 87: 523 (1990)). This was confirmed by similar studies by Pal et al. that demonstrated that glylala mutants of Gag are not processed to myristoylated proteins, and that CEM cells infected with virus carrying this mutation did not express viral cores or release virus particles (Pal et al., AIDS Res. Human Retrovir. 6: 721 (1990)). Furthermore, supernatant from these cells was not infectious. Others have clearly demonstrated that the non-myristoylated Gag mutants fail to support budding from the cell surface (Freed et al., J Virol. 68: 5311 (1994)), and that this is associated with impaired association of Gag products with “barges” in the plasma membrane (Linderwasser and Resh, J. Virol. 75: 7913 (2001)). In all, there has been clear demonstration by multiple research groups that disruption of Gag myristoylation very effectively blocks the replication of HIV and its release from infected cells.

A second HIV protein that requires N-myristoylation for activity is the Nef protein that regulates the virulence of the virus. Mutation of the N-terminal glycine residue of Nef blocks myristoylation and prevents it from associating with lipid rafts in the plasma membrane (Kaminchik et al., AIDS Res. Human Retrovir. 10: 1003 (1994); Wang et al., Proc. Natl. Acad. Sci. USA 97: 394 (2000)), abolishes its incorporation into HIV particles (Welker et al., J. Virol. 72: 8833 (1998)) and prevents it from down-regulating the expression of MHC-1 and CD4 proteins (Peng and Robert-Guroff, Immunol. Lett. 78: 195 (2001)). Since Nef is critical for high-titer virus replication in vivo, pharmacological inhibition of its myristoylation could be highly effective in blocking the progression of AIDS. It is important to recognize that the myristoylation of the HIV proteins Gag and Nef is accomplished by the host, i.e. human, NMT as the virus does not encode a protein with this activity. Therefore, attempts to pharmacologically manipulate HIV replication must center on the development of inhibitors of human NMT.

Several studies have examined the effects of myristic acid analogs on protein myristoylation and function (Boutin et al., Eur. J Biochem. 214: 853 (1993); Cordo et al., Microbes Infect. 1: 609 (1999); Heuckeroth and Gordon, Proc. Natl. Acad. Sci. USA 86: 5262 (1989)). These analogs act as alternative substrates for NMT and are transferred to the substrate proteins. In most cases, this results in a less hydrophobic modification of the protein, and this disrupts its targeting to lipid rafts in the plasma membrane. Studies of the effects of myristate analogs on the replication of HIV have consistently validated NMT as a target for the development of anti-AIDS drugs. For example, a variety of analogs of myristic acid have been shown to inhibit HIV replication in cell culture systems (Bryant et al., Proc. Natl. Acad. Sci. USA 88: 2055 (1991); Lindwasser and Resh, Proc. Natl. Acad. Sc.i USA 99: 13037 (2002)). Importantly, studies by Bryant et al. demonstrated synergistic inhibition of HIV replication when cells were treated with combinations of a myristate analog and the reverse transcriptase inhibitor AZT (Bryant et al., Proc. Natl. Acad. Sci. USA 88: 2055 (1991)). Additionally, the inhibitory effects of the myristate analogs were much more prolonged than the effects of AZT. There are few studies of the effects of NMT inhibitors (distinguished from alternate substrates) on HIV replication. In one case, N-myristoyl glycinal diethylacetal was shown to inhibit Gag myristoylation in MT-2 and CEM/LAV cells, and to substantially inhibit HIV replication in MT-4 cells (Tashiro et al., Biochem. Biophys. Res. Commun. 165: 1145 (1989)). Similarly, several serinal-based NMT inhibitors have been shown to suppress HIV replication in infected CEM cells (Takamune et al., IUBMB Life 48: 311 (1999)).

Intriguing studies by the Shoji lab demonstrated that infection of CEM cells with HIV results in a progressive decrease in the expression of NMT by the cells (Takamune et al., FEBS Lett. 506: 81 (2001)). This is associated with markedly increased cytotoxicity of the serinal NMT inhibitors toward HIV-infected CEM cells as compared to uninfected CEM cells (Takamune et al., FEBS Lett. 527: 138 (2002)). This indicates that NMT inhibitors can act as selective cytotoxins for HIV-infected lymphocytes, and so may be effective in destroying HIV reservoirs in latently infected cells.

To date, drug development efforts relating to NMT have focused on the identification of antifungal agents. Since NMT is essential for growth in yeast and fungi, Gordon and coworkers designed and synthesized potent antifungal peptidomimetic and dipeptide analog inhibitors for C. albicans NMT (Nagarajan et al., J Med. Chem. 40: 1422 (1997)). These inhibitors were designed to not affect human NMT, and therefore are expected to have no effect on processing of oncogenic or viral proteins by human NMT. Studies discussed above indicate that myristic acid analogs and serinal-based compounds can be effective for inhibiting HIV replication in tissue culture systems; however, these compounds are not suitable for clinical use because of their poor pharmacokinetic properties. Thus, the need remains for small molecule inhibitors of human NMT to be used as therapeutic agents. We report here the identification of novel chemotypes i.e. cyclohexyl-octahydro-pyrrolo[1,2-a]pyrazine (COPP) and methyl-octahydro-pyrrolo[1,2-a]pyrazine (MOPP) that inhibit human NMT with micromolar efficacy. Thus, these compounds are lead compounds for a new class of drugs.

### SUMMARY OF THE INVENTION

The invention is a pharmaceutical composition mainly for the treatment of hyperproliferative diseases or viral infection. The active ingredient of the composition is a non-lipid compound that inhibits the activity of human N-myristoyltransferase (NMT). Preferred compounds have a chemotype of cyclohexyl-octahydro-pyrrolo[1,2-a]pyrazine (COPP) or methyl-octahydro-pyrrolo[1,2-a]pyrazine chemotype (MOPP). Compositions useful in the practice of this invention contain compounds having the following formulae, including their pharmaceutically acceptable salts:

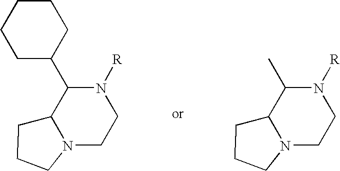

wherein R is selected from the group consisting of H, alkyl (C₁-C₁₅), cycloalkyl, cycloalkylalkyl, aryl, preferably phenyl, arylalkyl, preferably benzyl, heteroaryl, preferably pyridine, heteroarylalkyl, heterocycloalkyl, heterocycloalkylalkyl, halogen, haloalkyl, —OH, alkoxy, hydroxyalkyl, alkanoyl, —COOH, carboxamide, carbazole, mono or dialkylaminocarboxamide, —SH, —S-alkyl, —CF₃, —OCF₃, —NO₂, —NH₂, —CO₂R₈, —OC(O)R₈, carbamoyl, mono or dialkylcarbamoyl, mono- or dialkylamino, aminoalkyl, mono- or dialkylaminoalkyl, thiocarboxamide, or mono or dialkylthiocarboxamide;

wherein each of the above can be optionally substituted with up to 5 groups that are independently alkyl (C₁-C₆), halogen, haloalkyl, —CF₃, —OCF₃, —OH, alkoxy, hydroxyalkyl, —CN, —CO₂H, —SH, —S-alkyl, —NO₂, or —NR′R″, wherein R′ and R″ are independently H or alkyl (C₁-C₆).

The present invention further provides methods for using these compositions in the therapy of hyperproliferative disease, including cancer, atherosclerosis, restenosis and psoriasis, osteoporosis or viral infection by administering an effective amount of at least one of the compositions described herein to a patient in need of such treatment.

### DESCRIPTION OF THE DRAWINGS

FIG. 1 shows the activity of purified GST-NMT expressed in E. coli. The NMT assay was conducted in the absence of recombinant NMT (left two columns) or in the presence of vehicle alone (DMSO) or a known NMT inhibitor (myristoylphenylalanine). Values represent the mean NMT activity in triplicate samples of a representative experiment. Error bars represent the SEM of duplicate measurements.

FIG. 2 shows transient transfections of N-myristoylated GFP in monkey kidney cells in the presence of vehicle alone (DMSO), 2-hydroxymyristic acid or compound 537.

### DESCRIPTION OF THE PREFERRED EMBODIMENTS

The present invention provides pharmaceutical compositions and methods for their use in the treatment of disease. The chemical compounds therein and pharmaceutical compositions of the present invention may be useful in the therapy of hyperproliferative disease, osteoporosis or viral infection.

The term “alkoxy” represents an alkyl group of indicated number of carbon atoms attached to the parent molecular moiety through an oxygen bridge. Examples of alkoxy groups include, for example, methoxy, ethoxy, propoxy and isopropoxy.

The term “alkyl” includes those alkyl groups of a designed number of carbon atoms. Alkyl groups may be straight, or branched. Examples of “alkyl” include methyl, ethyl, propyl, isopropyl, butyl, iso-, sec- and tert-butyl, pentyl, hexyl, heptyl, 3-ethylbutyl, and the like.

The term “aryl” refers to an aromatic hydrocarbon ring system containing at least one aromatic ring. The aromatic ring may optionally be fused or otherwise attached to other aromatic hydrocarbon rings or non-aromatic hydrocarbon rings. Examples of aryl groups include, for example, phenyl, naphthyl, 1,2,3,4-tetrahydronaphthalene and biphenyl. Preferred examples of aryl groups include phenyl and naphthyl.

The term “cycloalkyl” refers to a C₃-C₈ cyclic hydrocarbon. Examples of cycloalkyl include cyclopropyl, cyclobutyl, cyclopentyl, cyclohexyl, cycloheptyl and cyclooctyl.

The term “cycloalkylalkyl,” as used herein, refers to a C₃-C₇ cycloalkyl group attached to the parent molecular moiety through an alkyl group, as defined above. Examples of cycloalkylalkyl groups include cyclopropylmethyl and cyclopentylethyl.

The terms “halogen” or “halo” indicate fluorine, chlorine, bromine, or iodine.

The term “heterocycloalkyl,” refers to a non-aromatic ring system containing at least one heteroatom selected from nitrogen, oxygen, and sulfur. The heterocycloalkyl ring may be optionally fused to or otherwise attached to other heterocycloalkyl rings and/or non-aromatic hydrocarbon rings. Preferred heterocycloalkyl groups have from 3 to 7 members. Examples of heterocycloalkyl groups include, for example, piperazine, morpholine, piperidine, tetrahydrofuran, pyrrolidine, and pyrazole. Preferred heterocycloalkyl groups include piperidinyl, piperazinyl, morpholinyl, and pyrolidinyl.

The term “heteroaryl” refers to an aromatic ring system containing at least one heteroatom selected from nitrogen, oxygen, and sulfur. The heteroaryl ring may be fused or otherwise attached to one or more heteroaryl rings, aromatic or non-aromatic hydrocarbon rings or heterocycloalkyl rings. Examples of heteroaryl groups include, for example, pyridine, furan, thiophene, 5,6,7,8-tetrahydroisoquinoline and pyrimidine. Preferred examples of heteroaryl groups include thienyl, benzothienyl, pyridyl, quinolyl, pyrazinyl, pyrimidyl, imidazolyl, benzimidazolyl, furanyl, benzofuranyl, thiazolyl, benzothiazolyl, isoxazolyl, oxadiazolyl, isothiazolyl, benzisothiazolyl, triazolyl, tetrazolyl, pyrrolyl, indolyl, pyrazolyl, and benzopyrazolyl.

In certain situations, the compounds of this invention may contain one or more asymmetric carbon atoms, so that the compounds can exist in different stereoisomeric forms. These compounds can be, for example, racemates, chiral non-racemic or diastereomers. In these situations, the single enantiomers, i.e., optically active forms, can be obtained by asymmetric synthesis or by resolution of the racemates. Resolution of the racemates can be accomplished, for example, by conventional methods such as crystallization in the presence of a resolving agent; chromatography, using, for example a chiral HPLC column; or derivatizing the racemic mixture with a resolving reagent to generate diastereomers, separating the diastereomers via chromatography, and removing the resolving agent to generate the original compound in enantiomerically enriched form. Any of the above procedures can be repeated to increase the enantiomeric purity of a compound.

When the compounds described herein contain olefinic double bonds or other centers of geometric asymmetry, and unless otherwise specified, it is intended that the compounds include the cis, trans, Z- and E-configurations. Likewise, all tautomeric forms are also intended to be included.

In one embodiment, the pharmaceutical compositions of the present invention comprise a cyclohexyl-octahydro-pyrrolo[1,2-a]pyrazine (COPP) compound (I) of the formula:

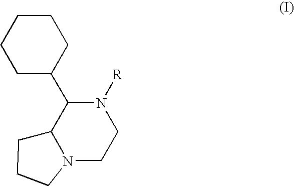

or a pharmaceutically acceptable salt thereof; with at least one pharmaceutically acceptable carrier or excipient, wherein R is selected from the group consisting of H, alkyl (C₁-C₁₅), cycloalkyl, cycloalkylalkyl, aryl, arylalkyl, heteroaryl, preferably pyridine, heteroarylalkyl, heterocycloalkyl, heterocycloalkylalkyl, halogen, haloalkyl, —OH, alkoxy, hydroxyalkyl, alkanoyl, —COOH, carboxamide, carbazole, mono or dialkylaminocarboxamide, —SH, —S-alkyl, —CF₃, —OCF₃, —NO₂, —NH₂, —CO₂R₈, —OC(O)R₈, carbamoyl, mono or dialkylcarbamoyl, mono- or dialkylamino, aminoalkyl, mono- or dialkylaminoalkyl, thiocarboxamide, or mono or dialkylthiocarboxamide;

wherein each of the above can be optionally substituted with up to 5 groups that are independently alkyl (C₁-C₆), halogen, haloalkyl, —CF₃, —OCF₃, —OH, alkoxy, hydroxyalkyl, —CN, —CO₂H, —SH, —S-alkyl, —NO₂, or —NR′R″, wherein R′ and R″ are independently H or alkyl (C₁-C₆).

In another embodiment, the pharmaceutical compositions of the present invention comprise a methyl-octahydro-pyrrolo[1,2-a]pyrazine (MOPP) compound (II) of the formula:

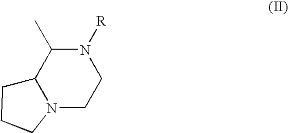

or a pharmaceutically acceptable salt thereof; with at least one pharmaceutically acceptable carrier or excipient, wherein R is selected from the group consisting of H, alkyl (C₁-C₁₅), cycloalkyl, cycloalkylalkyl, aryl, arylalkyl, heteroaryl, preferably pyridine, heteroarylalkyl, heterocycloalkyl, heterocycloalkylalkyl, halogen, haloalkyl, —OH, alkoxy, hydroxyalkyl, alkanoyl, —COOH, carboxamide, carbazole, mono or dialkylaminocarboxamide, —SH, —S-alkyl, —CF₃, —OCF₃, —NO₂, —NH₂, —CO₂R₈, —OC(O)R₈, carbamoyl, mono or dialkylcarbamoyl, mono- or dialkylamino, aminoalkyl, mono- or dialkylaminoalkyl, thiocarboxamide, or mono or dialkylthiocarboxamide;

wherein each of the above can be optionally substituted with up to 5 groups that are independently alkyl (C₁-C₆), halogen, haloalkyl, —CF₃, —OCF₃, —OH, alkoxy, hydroxyalkyl, —CN, —CO₂H, —SH, —S-alkyl, —NO₂, or —NR′R″, wherein R′ and R″ are independently H or alkyl (C₁-C₆).

In a more preferred embodiment, the pharmaceutical compositions of the present invention comprise a cyclohexyl-octahydro-pyrrolo[1,2-a]pyrazine (COPP) compound of formula (I) wherein R is selected from the group comprising: cyclohexyl, 3-pyridinyl, 2-methylbenzyl, 4-methoxybenzyl, 4-methylsulfanylbenzyl, 4-methoxycarbonylbenzyl, 4-trifluoromethylbenzyl, 2-fluorobenzyl, 2-nitrobenzyl, 4-methoxy-3-methylbenzyl, 3,4-dimethoxybenzyl, 3,4-dichlorobenzyl, 2,6-dichlorobenzyl, 3,4-difluorobenzyl, 2,6-difluorobenzyl, 4-methoxy-2,5-dimethylbenzyl, 5-bromo-2,4-dimethoxybenzyl, 2-bromo-4,5-dimethoxybenzyl, 2-nitro-4,5-dimethoxybenzyl, 2-naphthalenyl, 4-methoxy-naphthalene-1-ylmethyl, 2-methoxy-naphthalene-1-ylmethyl, 2-nitro-benzo[1,3]dioxol-1-ylmethyl, 9-ethyl-9H-carbazole-3-ylmethyl, 10b,10c-dihydro-pyrene-1-ylmethyl, 6-methyl-chromene-4-one-3-ylmethyl, 4-bromo-thiophen-2-ylmethyl, 5-(2-nitro-phenyl)-furan-2-ylmethyl, 5-(3-nitro-phenyl)-furan-2-ylmethyl, 5-(4-nitro-phenyl)-furan-2-ylmethyl, 2-(2,6,6-trimethyl-cyclohex-1-enyl)-ethyl, 3-(2-methoxy-phenyl)-allyl, 3-(4-chlorophenoxyl)-benzyl, and 2-(10-chloro-anthracen-9-yl)-methyl.

In another more preferred embodiment, the pharmaceutical compositions of the present invention comprise a methyl-octahydro-pyrrolo[1,2-a]pyrazine (MOPP) compound of formula (II) wherein R is selected from the group comprising: cyclohexyl, 3-pyridinyl, 2-methylbenzyl, 4-methoxybenzyl, 4-methylsulfanylbenzyl, 4-methoxycarbonylbenzyl, 4-trifluoromethylbenzyl, 2-fluorobenzyl, 2-nitrobenzyl, 4-methoxy-3-methylbenzyl, 3,4-dimethoxybenzyl, 3,4-dichlorobenzyl, 2,6-dichlorobenzyl, 3,4-difluorobenzyl, 2,6-difluorobenzyl, 4-methoxy-2,5-dimethylbenzyl, 5-bromo-2,4-dimethoxybenzyl, 2-bromo-4,5-dimethoxybenzyl, 2-nitro-4,5-dimethoxybenzyl, 2-naphthalenyl, 4-methoxy-naphthalene-1-ylmethyl, 2-methoxy-naphthalene-1-ylmethyl, 2-nitro-benzo[1,3]dioxol-1-ylmethyl, 9-ethyl-9H-carbazole-3-ylmethyl, 10b, 10c-dihydro-pyrene-1-ylmethyl, 6-methyl-chromene-4-one-3-ylmethyl, 4-bromo-thiophen-2-ylmethyl, 5-(2-nitro-phenyl)-furan-2-ylmethyl, 5-(3-nitro-phenyl)-furan-2-ylmethyl, 5-(4-nitro-phenyl)-furan-2-ylmethyl, 2-(2,6,6-trimethyl-cyclohex-1-enyl)-ethyl, 3-(2-methoxy-phenyl)-allyl, 3-(4-chlorophenoxyl)-benzyl, and 2-(10-chloro-anthracen-9-yl)-methyl.

Pharmaceutically acceptable salts of the chemical compounds of the present invention, which also have NMT inhibitory activity (e.g., the hydrochloride or sodium salts), may be prepared following procedures that are familiar to those skilled in the art.

The pharmaceutical compositions of the present invention comprise one or more of the NMT inhibitory compounds of the present invention, as active ingredients, in combination with a pharmaceutically acceptable carrier, medium, or auxiliary agent.

The pharmaceutical compositions of the present invention may be prepared in various forms for administration, including tablets, caplets, pills or dragees, or can be filled in suitable containers, such as capsules, or, in the case of suspensions, filled into bottles. As used herein “pharmaceutically acceptable carrier medium” includes any and all solvents, diluents, or other liquid vehicle; dispersion or suspension aids; surface active agents; preservatives; solid binders; lubricants and the like, as suited to the particular dosage form desired. Various vehicles and carriers used in formulating pharmaceutical compositions and known techniques for the preparation thereof are disclosed in Remington's Pharmaceutical Sciences (A. Osol et al. eds., 15th ed. 1975). Except insofar as any conventional carrier medium is incompatible with the chemical compounds of the present invention, such as by producing any undesirable biological effect or otherwise interacting in a deleterious manner with any other component of the pharmaceutical composition, the use of the carrier medium is contemplated to be within the scope of this invention.

In the pharmaceutical compositions of the present invention, the active agent may be present in an amount of at least 1% and not more than 95% by weight, based on the total weight of the composition, including carrier medium or auxiliary agents. Preferably, the proportion of active agent varies between 1% to 70% by weight of the composition. Pharmaceutical organic or inorganic solid or liquid carrier media suitable for enteral or parenteral administration can be used to make up the composition. Gelatin, lactose, starch, magnesium, stearate, talc, vegetable and animal fats and oils, gum polyalkylene glycol, or other known excipients or diluents for medicaments may all be suitable as carrier media.

The pharmaceutical compositions of the present invention may be administered using any amount and any route of administration effective for increasing the therapeutic efficacy of drugs. Thus the expression “therapeutically effective amount,” as used herein, refers to a sufficient amount of the NMT inhibitory agent to provide the desired effect against target cells. The exact amount required will vary from subject to subject, depending on the species, age, and general condition of the subject; the particular NMT inhibitory agent; its mode of administration; and the like.

The pharmaceutical compositions of the present invention are preferably formulated in dosage unit form for ease of administration and uniformity of dosage. “Dosage unit form,” as used herein, refers to a physically discrete unit of therapeutic agent appropriate for the animal to be treated. Each dosage should contain the quantity of active material calculated to produce the desired therapeutic effect either as such, or in association with the selected pharmaceutical carrier medium. Typically, the pharmaceutical composition will be administered in dosage units containing from about 0.1 mg to about 10,000 mg of the agent, with a range of about 1 mg to about 1000 mg being preferred.

The pharmaceutical compositions of the present invention may be administered orally or paternally, such as by intramuscular injection, intraperitoneal injection, or intravenous infusion. The pharmaceutical compositions may be administered orally or parenterally at dosage levels of about 0.1 to about 1000 mg/kg, and preferably from about 1 to about 100 mg/kg, of animal body weight per day, one or more times a day, to obtain the desired therapeutic effect.

Although the pharmaceutical compositions of the present invention can be administered to any subject that can benefit from the therapeutic effects of the compositions, the compositions are intended particularly for the treatment of diseases in humans.

The pharmaceutical compositions of the present invention will typically be administered from 1 to 4 times a day, so as to deliver the daily dosage as described herein. Alternatively, dosages within these ranges can be administered by constant infusion over an extended period of time, usually 1 to 96 hours, until the desired therapeutic benefits have been obtained. However, the exact regimen for administration of the chemical compounds and pharmaceutical compositions described herein will necessarily be dependent on the needs of the animal being treated, the type of treatments being administered, and the judgment of the attending physician.

The pharmaceutical compositions of the present invention can be used in various protocols for treating animals, including humans. In one embodiment of the methods of the present invention, NMT in target cells or tissues in an animal undergoing chemotherapy is inhibited by administering to the animal a pharmaceutical composition of the present invention in an amount effective to inhibit NMT in the target cells or tissues of the animal.

In a particularly preferred embodiment of the use of the pharmaceutical compositions of the present invention, the compositions can be used in a method for treating hyperproliferative cells in a patient requiring such treatment, by administering the composition to a patient in an amount effective to inhibit the proliferation of said cells. For example, these pharmaceutical compositions can be used in a method for treating tumor cells in a patient requiring such treatment. This method would involve administering to a cancer patient a composition of the present invention as described above in an amount effective to prevent the proliferation of such cells.

In another particularly preferred embodiment of the use of the pharmaceutical compositions of the present invention, the compositions can be used in a method for treating viral infection in a patient requiring such treatment, by administering the composition to a patient in an amount effective to inhibit the proliferation of said virus. For example, these pharmaceutical compositions can be used in a method for treating an AIDS patient requiring such treatment. This method would involve administering to the patient a composition of the present invention as described above in an amount effective to prevent the proliferation of the human immunodeficiency virus.

In another preferred embodiment of the use of the pharmaceutical compositions of the present invention, the compositions can be used in a method for treating hyperproliferating cells present in lesions in atherosclerosis or restenosis. In both cases, inhibition of the proliferation of smooth muscle cells at the site of these lesions is expected to be useful in the treatment of the disease.

In another preferred embodiment of the use of the pharmaceutical compositions of the present invention, the compositions can be used in a method for treating hyperproliferating cells present in lesions in psoriasis. Inhibition of the proliferation of epidermal cells at the site of these lesions is expected to be useful in the treatment of the disease.

In another preferred embodiment of the use of the pharmaceutical compositions of the present invention, the compositions can be used in a method for treating bone loss in patients with osteoporosis.

In view of the beneficial effect of inhibiting NMT, as produced by the pharmaceutical compositions of the present invention, it is anticipated that these pharmaceutical compositions will be useful not only for therapeutic treatment following the onset of disease, but also for the prevention of disease in animals. The dosages described herein will be essentially the same whether the pharmaceutical compositions of the present invention are being administered for the treatment or prevention of disease.

While the invention has been particularly shown and described with reference to preferred embodiments thereof, it will be understood by those skilled in the art that various alterations in form and detail may be made therein without departing from the spirit and scope of the invention. In particular, the specific analogues used in the composition can vary significantly without departing from the discovered chemotype. Additionally, various pharmaceutically acceptable salts of these compounds are considered to be within the scope of this invention. Also, one skilled in the art of pharmaceutical compositions and delivery systems will understand that many common pharmaceutically acceptable ingredients including carriers and adjuvants, though not specifically mentioned here, can be included in the inventive composition with only routine experimentation. Similarly, a variety of modifications can be incorporated into the described assays for inhibition of NMT both in vitro and in intact cells, and these modifications are considered to be within the scope of the following claims.

The Examples, which follow, are illustrative of specific embodiments of the invention, and various uses thereof. They are set forth for explanatory purposes only, and are not to be taken as limiting the invention.

### EXAMPLE 1

Development of the NMT screening assay. An assay for screening for inhibitors of purified recombinant human N-myristoyltransferase has been established. The cDNA encoding enzymatically active human NMT-1 was subcloned into the pGEX bacterial expression evector, producing an NMT-glutathione-S-transferase4 (GST) fusion protein. This protein was expressed in E. coli using methods well known to those trained in the art, purified using a glutathione resin, and stored at −80° C. until use. Enzyme activity was measured using the recombinant NMT, a peptide of the sequence GNAASARRK-biotin, wherein amino acids are designated by their recognized single letter code and the terminal lysine residue is conjugated with biotin, [³H]-myristoyl-CoA, and a test compound or the solvent alone (DMSO). Reaction mixtures are incubated for a specific period of time in 96-well plates coated with streptavidin, which may be purchased from commercial sources including Sigma Chemical Company. The biotin-conjugated peptide binds strongly to the immobilized streptavidin thereby providing a solid phase support so that subsequent washes effectively remove [³H]-myristoyl-CoA that has not been covalently attached to the peptide through the action of NMT. The streptavidin-biotin interaction can then be disrupted with the addition of 0.1 M glycine, pH 2.8, and the solution containing [³H]myristoyl-peptide can be transferred to scintillation vials. Alternately, determination of the amount of [³H]myristoyl-peptide in the samples can be determined using scintillation proximity technology according to methods well known to those who practice the art. The amount of radioactivity in samples containing the test compound is then compared with the amount of radioactivity in control samples, such that a decrease in the amount of [³H]myristoyl-peptide indicates that the test compound inhibits NMT activity.

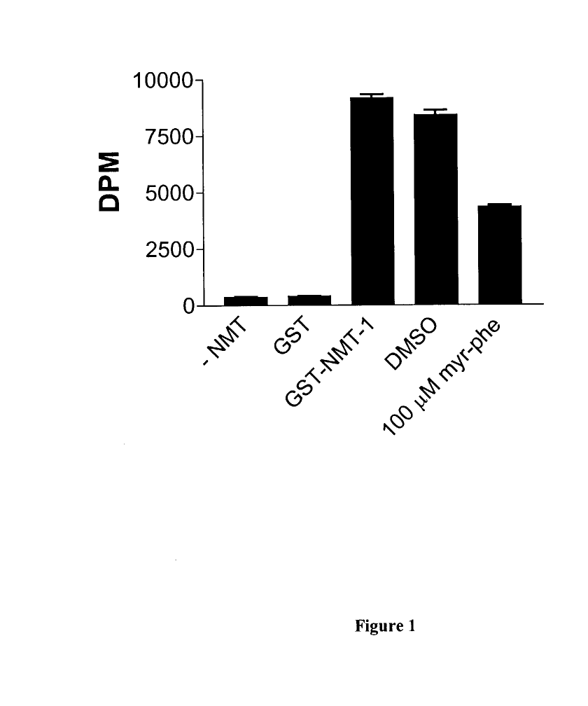

The results of a typical experiment are depicted in FIG. 1. As shown, there is a very low background level of non-specific binding of [³H]myristoyl-CoA to the plates (-NMT). Also, GSH column-purified lysate from E. coli expressing a control vector with GST alone shows no NMT activity. In contrast, addition of recombinant GST-NMT-1 results in very large increases in bound radioactivity, thereby providing a sensitive method of detecting inhibitors. The addition of DMSO, the solvent used in the chemical library, causes only a small decrease in NMT activity. A positive control, myristoylphenylalanine, was tested at the published IC₅₀ concentration of 100 μM (Boutin et al., Eur. J Biochem. 201: 257 (1991)) and does in fact inhibit the recombinant human NMT by approximately 50%. Therefore, a sensitive and robust method for screening for inhibitors of human NMT has been established.

### EXAMPLE 2

Identification of the COPP chemotype of NMT inhibitors. To facilitate screening of the compound library consisting of approximately 16,000 “drug-like” small molecules purchased from the ChemBridge Corporation (San Diego, Calif.), we used a combinatorial method in which GST-hNMT-l was simultaneously incubated with 8 compounds. Each compound was tested at a final concentration of 5 μg/ml, which corresponds to concentrations of 10-20 μM. Samples that inhibited NMT activity by at least 60% were then deconvoluted, where each of the 8 compounds was analyzed individually. This level of inhibition was observed in approximately 0.8% of the compounds tested.

Structural features of hits from the NMT inhibitor screen were compared. It was apparent that several NMT inhibitors share a common structural motif, i.e. a chemotype, consisting of a cyclohexyl-octahydro-pyrrolo[1,2-a]pyrazine moiety (COPP) as follows:

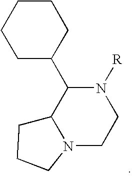

Additional inhibitory compounds contain a methyl-octahydro-pyrrolo[1,2-a]pyrazine moiety as follows:

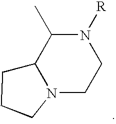

Compounds containing either moiety were individually tested in greater detail to determine their potencies for inhibiting human NMT. The IC₅₀ values, along with their differing R-groups, are listed in TABLE 1. Compound 537 demonstrated the highest potency (6 μM) for inhibition of NMT, while a number of other compounds demonstrated IC₅₀s below 100 μM, making them good lead compounds for further development. Additionally, 13 COPP compounds were poor inhibitors of NMT. In general, the methyl-octahydro-pyrrolo[1,2-a]pyrazine-containing compounds were less effective at inhibiting NMT than were the COPP-containing compounds; however, compounds 726 (2-[3-(4-chloro-phenoxy)-benzyl]-1-methyl-octahydro-pyrrolo[1,2-a]pyrazine) and 766 (2-(10-chloro-anthracen-9-yl)-methyl]-1-methyl-octahydro-pyrrolo[1,2-a]pyrazine) were moderate inhibitors of NMT, with IC₅₀s of 40±6 and 47±7 μM, respectively.

TABLE 1
Compound number, R substituent, and IC₅₀ values for inhibition of
recombinant human NMT and cytotoxicity toward T24 human bladder carcinoma cells
for members of the COPP family of inhibitors.

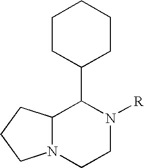

| | | NMT | |
|---|---|---|---|
| | | inhibition | Cytotoxicity |
| Compound | R | IC₅₀ (μM) | IC₅₀ (μM) |
| 624 | 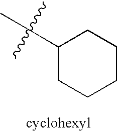 | 113 ± 7 | 1.6 |
| 522 | 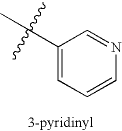 | 590 ± 127 | 400 |
| 524 | 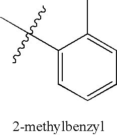 | 121 ± 8 | 2.4 |
| 486 | 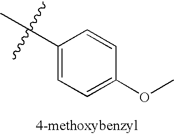 | 419 ± 30 | 8.1 |
| 515 | 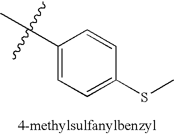 | 49 ± 13 | 1.9 |
| 607 | 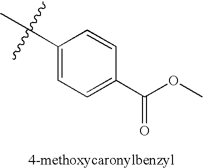 | 6400 ± 310 | 2.7 |
| 554 | 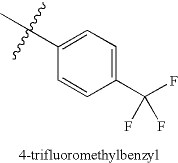 | 44 ± 6 | 1.3 |
| 508 | 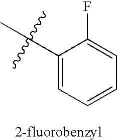 | >8000 | 3.5 |
| 518 | 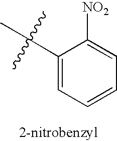 | >8000 | 4 |
| 561 | 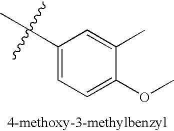 | 284 ± 46 | 1.8 |
| 502 | 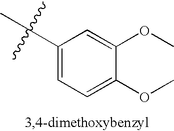 | 240 ± 23 | >500 |
| 498 | 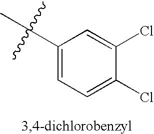 | 48 ± 5 | 1.6 |
| 576 | 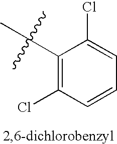 | 77 ± 13 | 1.3 |
| 558 | 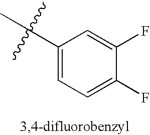 | 89 ± 16 | 6.5 |
| 601 | 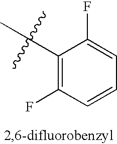 | 6000 ± 250 | 2 |
| 565 | 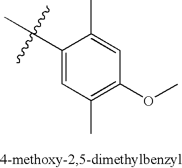 | 66 ± 13 | 1.8 |
| 548 | 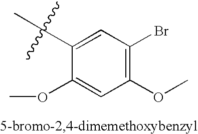 | 50 ± 2 | 1.9 |
| 605 | 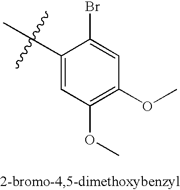 | 78 ± 1 | 2.5 |
| 553 | 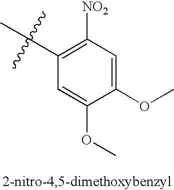 | 60 ± 6 | 5.6 |
| 542 | 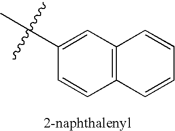 | 42 ± 5 | 1.3 |
| 621 | 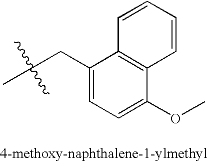 | 35 ± 5 | 0.7 |
| 622 | 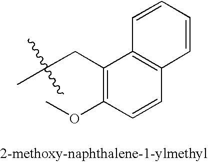 | 28 ± 8 | 1.5 |
| 543 | 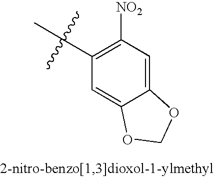 | 63 ± 15 | 5.8 |
| 537 | 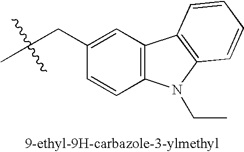 | 6 ± 1 | 1.8 |
| 629 | 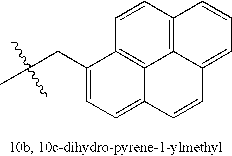 | 70 ± 8 | 3. |
| 591 | 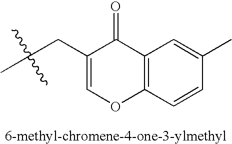 | >8000 | 9.7 |
| 555 | 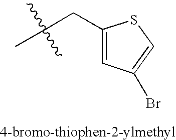 | >8000 | 1.2 |
| 615 | 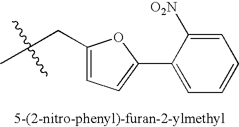 | 45 ± 2 | 1.4 |
| 616 | 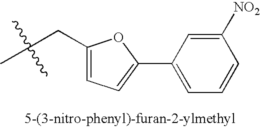 | 41 ± 1 | 2.1 |
| 617 | 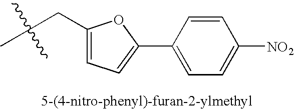 | 36 ± 4 | 4.6 |
| 638 | 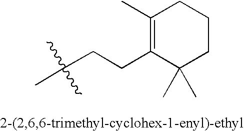 | 43 ± 4 | 1.4 |
| 640 | 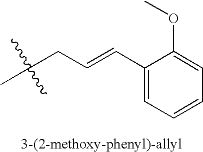 | 1600 ± 200 | 2.8 |

### EXAMPLE 3

Mechanism of inhibition of NMT by COPP compounds. The mechanism of inhibition by compound 537 was examined using peptide and myristoyl-CoA substrate competition experiments with GST-hNMT-l. The results of these studies are summarized in TABLE 2. In the absence of inhibitor, the K_{M} for myristoyl-CoA compares very well with the literature value of 2.6 μM (Rocque et al., J Biol. Chem. 268: 9964 (1993)). The affinity of the enzyme for the biotinylated peptide is somewhat lower than the affinity of hNMT for the soluble peptide GNAASARR (K_{M}=6 μM in Rocque et al., J Biol. Chem. 268: 9964 (1993)). However, it is well established that the affinity for the peptide is highly sequence-sensitive, so that K_{M}S >100 μM are not unusual. For studies of the ability of compound 537 to interact with the peptide-binding site of NMT, the peptide concentration was varied from 0 to 500 μM, while the myristoyl-CoA concentration was kept constant in the assays. The presence of compound 537 at its IC₅₀ concentration decreased the KM for the peptide relative to the solvent control. However, inhibition was overcome by the addition of excess peptide, as demonstrated by the similar Vₘₐₓ values, indicating that compound 537 acts as a competitive inhibitor with respect to the peptide substrate. To determine the effects of compound 537 on the myristoyl-CoA binding site, the concentration of myristoyl-CoA was varied from 0 to 100 μM, while the peptide concentration was held constant in the assays. As shown in TABLE 2, the K_{M} for myristoyl-CoA was unchanged by the presence of compound 537. In contrast, the Vₘₐₓ decreased in the presence of compound 537, indicating that it acts as a noncompetitive inhibitor with respect to the lipid substrate. Overall, these kinetic analyses indicate that compound 537, and most likely the rest of the COPP family, inhibit NMT activity by competing with the substrate peptide for binding to the enzyme.

TABLE 2
Michaelis-Menten parameters for competition assays with Compound 537.

| | | | Myristoyl-CoA | |
|---|---|---|---|---|
| | Peptide | | | Vₘₐₓ |
| | K_{M} (μM) | Vₘₐₓ (pmol/min) | K_{M} (μM) | (pmol/min) |
| DMSO | 181 ± 1 | 0.084 ± 0.012 | 3.0 ± 1.8 | 0.057 ± 0.005 |
| 537 | 252 ± 2 | 0.076 ± 0.006 | 2.6 ± 2.0 | 0.034 ± 0.001 |

### EXAMPLE 4

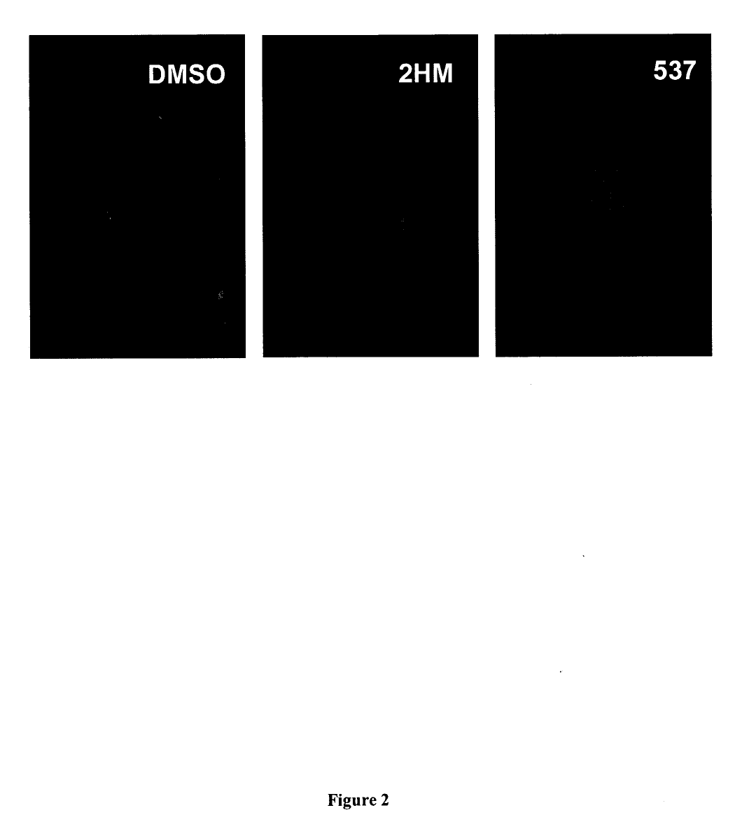

Cellular effects of COPP compounds. The studies described above demonstrate the ability of COPP-containing compounds to inhibit purified human NMT. To determine their efficacy in intact cells, a novel assay was developed in which the N-terminus of enhanced green fluorescent protein (GFP) was modified to allow its myristoylation (N-myr-GFP) by endogenous NMT. To begin, the myristoylation sequence MGCVQCKTKLTEER was added to the N-terminus of green fluorescent protein (N-myr-GFP) using techniques familiar to those practiced in the art. Since GFP is intrinsically fluorescent, the sites of its intracellular distribution can be visualized using fluorescence microscopy. When a plasmid containing the N-myr-GFP sequence is transfected into CV-1 cells, the GFP encoded by this construct is myristoylated by endogenous NMT causing it to localize to the plasma membrane, as indicated by the bright fluorescence at the cell periphery (FIG. 2). Treatment of the N-myr-GFP-transfected cells with millimolar concentrations of a known, very low potency, inhibitor of NMT, 2-hydroxymyristic acid (2HM), resulted in redistribution of GFP to the cytosol. Similar effects are seen when the cells are treated with micromolar concentrations of compound 537, i.e. displacement of N-myr-GFP to the cytosol. The N-terminal sequence of the N-myr-GFP construct is different than the peptide substrate used in the screening assays, demonstrating that the COPP compounds can inhibit the myristoylation of a variety of proteins. This method provides evidence that the inhibitors detected in the screen with recombinant NMT can also block endogenous NMT activity in whole cells.

### EXAMPLE 5

Antiproliferative effects of compound 537. To determine the abilities of NMT inhibitors to affect the growth of hyperproliferative cells, cytotoxicity assays were performed using the T24 human bladder carcinoma cell line. Cytotoxicity toward tumor cells growing in culture is a strong indicator of antitumor activity in vivo. In these assays, T24 cells growing in tissue culture in the presence of 10% fetal bovine serum are treated with varying concentrations of a test compound for 48 hours. At the end of the treatment period, the number of surviving tumor cells is quantified using any of a variety of methods well known in the field. In these, experiments, the number of surviving cells was determined using the sulforhodamine B binding assay. The percentage of surviving tumor cells at each concentration of the test compound is calculated and the concentration of compound that reduces the cell number by 50%, compared with samples that lack the test compound, is defined as the IC₅₀. Most of the methyl-octahydro-pyrrolo[1,2-a]pyrazine-containing compounds were non-toxic at doses up to at least 100 μM. However, the NMT-inhibiting compounds 726 and 766 demonstrated IC₅₀s of 1.5 and 4.2 μM, respectively. As demonstrated in TABLE 1, the COPP-containing compounds demonstrated a wide range of IC₅₀s, with most compounds having significant cytotoxicity at low micromolar concentrations. All of the compounds that inhibited NMT at micromolar levels shared similar cytotoxicity levels. This correlation suggests that these inhibitors are capable of entering whole cells causing inhibition of cell proliferation at micromolar levels.

Additionally, the effects of compound 537 on the proliferation of a panel of human tumor cell lines were determined. As indicated in TABLE 3, compound 537 caused dose-dependent inhibition of the proliferation of a variety of tumor cell lines. The IC₅₀s for compound 537 fell within a relatively narrow concentration range (3.7-14.8 μM), suggesting that this class of compound may be useful for the treatment of tumors that derive from a variety of tissues. Of interest, colon-derived HT29 cells were most sensitive to compound 537, and previous work has indicated a close association of activation of Src (which requires myristoylation for activity) and colon cancer (Raju et al., Exp. Cell Res. 235: 145 (1997)). Overall, the data indicate that compound 537, and likely related COPPs, have excellent potential utility as anticancer agents.

TABLE 3
Antiproliferative activity of compound 537 toward human tumor cell lines.
Values represent the mean ± SEM of 4 experiments.

| Cell Line | Tissue of origin | IC₅₀ (μM) |
|---|---|---|
| DU145 | Prostate | 14.8 ± 2.9 |
| HepG2 | Liver | 4.4 ± 1.2 |
| HT29 | Colon | 3.7 ± 0.6 |
| MCF-7 | Breast | 8.0 ± 0.9 |
| MDA-MB-231 | Breast | 11.9 ± 1.3 |
| Panc-1 | Pancreas | 12.6 ± 1.3 |
| SKOV3 | Ovary | 13.2 ± 3.6 |
| A-498 | Kidney | 5.8 ± 1.4 |

### EXAMPLE 6

Synthesis of COPP compounds. As shown by Leditschke and others (Leditschke, Chem. Ber. 86: 123 (1953); Likhosherstov and Skoldinov, Zhurnal Organicheskoi Khimii 22: 2610 (1986); Lopez-Rodriguez et al., J. Med. Chem. 44: 186 (2001)), an extensive and diverse set of COPP-containing compounds can be efficiently synthesized through four steps, as outlined below. Commercially available furan-2-carbonitrile (1) is condensed with the cyclohexyl Grignard compound (2) in dry acetone to form the 2-acylfuran (3). This is then refluxed in 90% aqueous ethylenediamine to produce 4. Reduction with LiAlH₄ followed by Pd-catalyzed hydrogenation produces 1-cyclohexyl-octahydropyrrolo[1,2-a]pyrazine (5), which is the central scaffold for the synthesis of the COPP library. Compound 5 can be reacted with a broad range of substituted aryl or alkyl bromides in DMF to generate the 2-substituted COPP library. Additional diversity can be introduced by varying the Grignard reagent (2) which will result in substituents other than cyclohexyl at position 1.

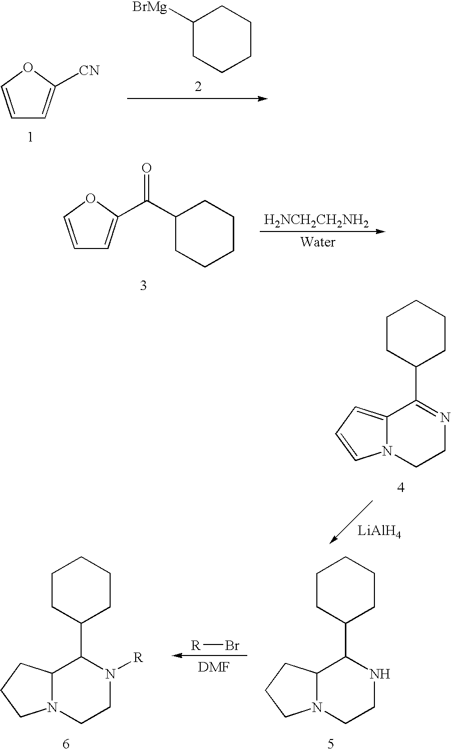

We claim:

1. A therapeutic composition comprising a compound of the formula: 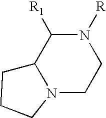
wherein: R is selected from the group consisting of H, alkyl (C₁-C₁₅), cycloalkyl, cycloalkylalkyl, aryl, arylalkyl, heteroaryl, preferably pyridine, heteroarylalkyl, heterocycloalkyl, heterocycloalkylalkyl, halogen, haloalkyl, —OH, alkoxy, hydroxyalkyl, alkanoyl, —COOH, carboxamide, carbazole, mono or dialkylaminocarboxamide, —SH, —S-alkyl, —CF₃, —OCF₃, —NO₂, —NH₂, —CO₂R₈, —OC(O)R₈, carbamoyl, mono or dialkylcarbamoyl, mono- or dialkylamino, aminoalkyl, mono- or dialkylaminoalkyl, thiocarboxamide, or mono or dialkylthiocarboxamide;
wherein each of the above can be optionally substituted with up to 5 groups that are independently alkyl (C₁-C₆), halogen, haloalkyl, —CF₃, —OCF₃, —OH, alkoxy, hydroxyalkyl, —CN, —CO₂H, —SH, —S-alkyl, —NO₂, or —NR′R″, wherein R′ and R″ are independently H or alkyl (C₁-C₆); and
R₁ is cyclohexyl or methyl;
or a pharmaceutically acceptable salt thereof; with at least one pharmaceutically acceptable carrier or excipient.

2. A process for treating a hyperproliferative disease comprising delivering to a patient a therapeutic composition according to claim 1 in a pharmaceutically acceptable carrier.

3 The process of claim 2 wherein said hyperproliferative disease is selected from the group consisting of cancer, atherosclerosis, restenosis and psoriasis.

4. A process for treating osteoporosis comprising delivering to a patient a therapeutic composition according to claim 1 in a pharmaceutically acceptable carrier.

5. A process for treating a viral infection comprising delivering to a patient a therapeutic composition according to claim 1 in a pharmaceutically acceptable carrier.

6. The process of claim 5 wherein said viral infection is caused by a Human Immunodeficiency Virus or a Human T-Cell Leukemia Virus.

7. A therapeutic composition comprising a compound of the formula: 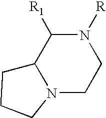
wherein: R is selected from the group consisting of cyclohexyl, 3-pyridinyl, 2-methylbenzyl, 4-methoxybenzyl, 4-methylsulfanylbenzyl, 4-methoxycarbonylbenzyl, 4-trifluoromethylbenzyl, 2-fluorobenzyl, 2-nitrobenzyl, 4-methoxy-3-methylbenzyl, 3,4-dimethoxybenzyl, 3,4-dichlorobenzyl, 2,6-dichlorobenzyl, 3,4-difluorobenzyl, 2,6-difluorobenzyl, 4-methoxy-2,5-dimethylbenzyl, 5-bromo-2,4-dimethoxybenzyl, 2-bromo-4,5-dimethoxybenzyl, 2-nitro-4,5-dimethoxybenzyl, 2-naphthalenyl, 4-methoxy-naphthalene-1-ylmethyl, 2-methoxy-naphthalene-1-ylmethyl, 2-nitro-benzo[1,3]dioxol-1-ylmethyl, 9-ethyl-9H-carbazole-3-ylmethyl, 10b, 10c-dihydro-pyrene-1-ylmethyl, 6-methyl-chromene-4-one-3-ylmethyl, 4-bromo-thiophen-2-ylmethyl, 5-(2-nitro-phenyl)-furan-2-ylmethyl, 5-(3-nitro-phenyl)-furan-2-ylmethyl, 5-(4-nitro-phenyl)-furan-2-ylmethyl, 2-(2,6,6-trimethyl-cyclohex-1-enyl)-ethyl, 3-(2-methoxy-phenyl)-allyl, 3-(4-chlorophenoxyl)-benzyl, and 2-(10-chloro-anthracen-9-yl)-methyl; and
R₁ is cyclohexyl or methyl;
or a pharmaceutically acceptable salt thereof; with at least one pharmaceutically acceptable carrier or excipient.

8. A process for treating a hyperproliferative disease comprising delivering to a patient a therapeutic composition according to claim 7 in a pharmaceutically acceptable carrier.

9. The process of claim 8 wherein said hyperproliferative disease is selected from the group consisting of cancer, atherosclerosis, restenosis and psoriasis.

10. A process for treating osteoporosis comprising delivering to a patient a therapeutic composition according to claim 7 in a pharmaceutically acceptable carrier.

11. A process for treating a viral infection comprising delivering to a patient a therapeutic composition according to claim 7 in a pharmaceutically acceptable carrier.

12. The process of claim 11 wherein said viral infection is caused by a Human Immunodeficiency Virus or a Human T-Cell Leukemia Virus.
